(19) United States
(12) Patent Application Publication
(10) Pub. No.: US 2011/0021640 A1
(43) Pub. Date: Jan. 27, 2011

(54) Mechanism-based biochemical standardization of resveratrol products and their uses thereof

(76) Inventor: Kailash Chandra Agarwal, Durham, NC (US)

Correspondence Address: KAILASH CHANDRA AGARWAL, 8613 EAGLEVIEW DRIVE, DURHAM, NC, 27713, US

(21) Appl. No.: 12/804,633

(22) Filed: Jul. 26, 2010

Related U.S. Application Data
(60) Provisional application No. 61/271,863, filed on Jul. 27, 2009.

(51) Int. Cl. A61K 31/05; A61P 7/02; C12Q 1/34

(52) U.S. Cl. 514/733; 435/18

(57) ABSTRACT

The present invention pertains to methods (a) Inhibition of adenosine deaminase by resveratrol products including botanical extracts containing resveratrol and/or oxyresveratrol; (b) Biochemical Standardization of these products as Enzyme Inhibitory Units (EIU) per mg or gram; and (c) Their uses thereof.

This application is being filed on Jul. 26, 2010, as a PCT International Patent Application in the name of Kailash Chandra Agarwal, a citizen of the U.S., applicant for the designation of the U.S. only, and claims priority to U.S. Provisional Patent Application Ser. No. 61/271,863 filed on Jul. 27, 2009.

### FIELD OF INVENTION

The present invention pertains to methods for inhibition of adenosine deaminase enzyme by resveratrol products including oxyresveratrol, biochemical standardization, and their uses thereof.

### BACKGROUND

### Resveratrol

Resveratrol (structural formula included below), a polyphenolic phytoalexin (3,5,4′-trihydroxystilbene) is produced naturally by several plants including red grapes, peanuts, blueberries, and mulberry wood. In grapes, resveratrol is primarily present in the skin. Most red wines contain resveratrol in quantities ranging 0.2-5.8 mg/liter depending on the grape variety and process of fermentation. Resveratrol is sold as a nutritional supplement derived primarily from Polygonum Cuspidatum “the Japanese Knotweed.” Resveratrol products have a number of beneficial health effects, such as anticancer, antiviral, neuroprotective, anti-aging, anti-inflammatory, blood-sugar-lowering, beneficial cardiovascular effects, and life-prolonging effects. Some suggest consumption of red wine, which contains resveratrol, may explain the “French paradox” that the incidence of coronary heart diseases is relatively low in southern France despite high dietary intake of saturated fats.

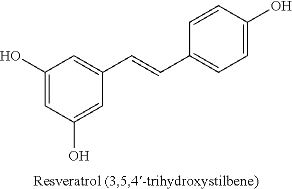

### Oxyresveratrol

Oxyresveratrol is structurally similar to resveratrol with an additional hyroxyl (OH) group at 5′-position (2,3′,4,5′-tetrahydroxystilbene) as shown below. Oxyresveratrol is also present in several botanicals such as mulberry (Morus alba Linne), Schoenocaulon officinale, Gnetum hainanense, and Artocarpus lakoocha. It is contained in high amount in mulberry wood in comparison to resveratrol, and extracted from mulberry (Morus alba) wood. Oxyresveratrol similar to resveratrol demonstrates strong antioxidant activity. However, oxyresveratrol is more effective as free radical scavenger than resveratrol. In addition, oxyresveratrol possesses a much stronger inhibitory activity of tyrosinase enzyme than resveratrol. As discussed later, this invention has also found that mulberry extract that contains oxyresveratrol is 2-fold more inhibitory of the enzyme, adenosine deaminase than resveratrol.

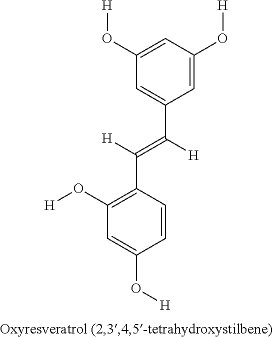

Some important work in relation to resveratrol products including oxyresveratrol is listed in the following publications:
1. Celotti E and others. Resveratrol content of some wines obtained from dried Valpolicella grapes: Recioto and Amarone. Journal of Chromatography A 730(1-2): 47-52, 1996.
2. Celotti E and others. Resveratrol content of some wines obtained from dried Valpolicella grapes: Recioto and Amarone. Journal of Chromatography A 730(1-2): 47-52, 1996.
3. A D, Mehta R G, Moon R C, Pezzuto J M, “Cancer chemopreventive activity of re Bertelli A and others. Plasma and tissue resveratrol concentrations and pharmacological activity. Drugs under Experimental and Clinical Research 24:133-138, 1997.
4. Kopp P. Resveratrol, a phytoestrogen found in red wine. A possible explanation for the conundrum of the ‘French paradox’? European Journal of Endocrinology 138:619-620, 1998.
5. Bertelli A and others. Plasma and tissue resveratrol concentrations and pharmacological activity. Drugs under Experimental and Clinical Research 24:133-138, 1998.
6. Hung L M, Chen J K, Huang S S, Lee R S, Su M J, “Cardioprotective effect of resveratrol, a natural antioxidant derived from grapes”. Cardiovasc. Res. 47 (3): 549-55, 2000.
7. Howitz K T, Bitterman K J, Cohen H Y, Lamming D W, Lavu S, Wood J G, Zipkin R E, Chung P, Kisielewski A, Zhang L L, Scherer B, Sinclair DA, “Small molecule activators of sirtuins extend Saccharomyces cerevisiae lifespan” (PDF). Nature 425: 191-196, 2003.
8. Wood J G, Rogina B, Lavu1 S, Howitz K, Helfand S L, Tatar M, Sinclair D, “Sirtuin activators mimic caloric restriction and delay aging in metazoans” (PDF). Nature 430: 686-9, 2004.
9. Kaeberlein M et al. Sir2-independent life span extension by calorie restriction in yeast. PLoS Biol. September; 2(9), 2004.
10. Farina A, Ferranti C, Marra C, “An improved synthesis of resveratrol”. Nat. Prod. Res. 20 (3): 247-52, 2006.
11. Valenzano D R, Terzibasi E, Genade T, Cattaneo A, Domenici L, Cellerino A “Resveratrol Prolongs Lifespan and Retards the Onset of Age-Related Markers in a Short-Lived Vertebrate.” Current Biology, February 7; 16 (3), 2006.
12. Baur J A, Pearson K J, Price N L, Jamieson H A, Lerin C, Kalra A, Prabhu W, Allard J S, Lopez-Lluch G, Lewis K, Pistell P J, Poosala S, Becker K G, Boss O, Gwinn D, Wang M, Ramaswamy S, Fishbein K W, Spencer R G, Lakatta E G, Le Couteur D, Shaw R J, Navas P, Puigserver P, Ingram D K, de Cabo R, Sinclair D A (2006). “Resveratrol improves health and survival of mice on a high-calorie diet”. Nature 444 (7117): 337-42, 2006
13. Wade, Nicholas, “Red Wine Ingredient Increases Endurance, Study Shows”. New York Times, Nov. 16, 2006.
14. See review: Baur J A, Sinclair D A, “Therapeutic potential of resveratrol: the in vivo evidence”. Nat Rev Drug Discov 5 (6): 493-506, 2006.
15. Stefani M, Markus M A, Lin R C, Pinese M, Dawes I W, Morris B J, “The effect of resveratrol on a cell model of human aging”. Ann N Y Acad Sci. 1114: 407-18, 2007.
16. Robb E L, Page M M, Wiens B E, Stuart J A, “Molecular mechanisms of oxidative stress resistance induced by resveratrol: Specific and progressive induction of MnSOD”. Biochem Biophys Res Commun. 367 (2): 406-12, 2008.
17. Elliott P J, Jirousek, M, “Sirtuins: Novel targets for metabolic disease”. Current Opinion in Investigational Drugs 9 (4): 1472-4472, 2008.
18. Shin N-H, Lee K S, Kang S-H, Min K R, Lee S-H, Kim, Y; Nat Prod Science: 3, 111-121 (1997).
19. Kim Y M, Yun J, Lee C-K, Lee H, Min K R, Kim Y; Oxyresveratrol and Hydroxystilbene Compounds: Inhibitory effect on tyrosinase and mechanism of action; J Biol Chem: 277, 16340-16344 (2002).
20. Lorenz P, Roychowdhury S, Engelmann M, Wolf G, Horn T F; Oxyresveratrol and resveratrol are potent antioxidants and free radicals scavengers: Nitric Oxide, 9, 64-76 (2003).
21. Tengamnuay P, Pengrungruangwong K, Pheansri I, Likhitwitayawuid K; Artocarpus lakoocha heartwood extract as a novel cosmetic ingredient and in vivo skin whitening activities: Intl J Cosmetic Science, 28, 269-276 (2006).
22. Qiu F, Komatsu K, Kawasaki K, Saito K, Yao X, Kano Y; A novel stilbene glucoside, oxyresveratrol 3′-O-beta-glucopyranoside from the root bark of Morus alba: Plant Med, 62, 559-561 (1996).
23. Chung K O, Kim B Y, Lee Y R, chung H Y, Park J H, Moon J O; In-vitro and in-vivo anti-inflammatory effect of oxyresveratrol from Morus alba L: J Pharm Pharmacol, 55, 1695-1700 (2003).
24. Oh H, Ko E K, Jun J Y, Oh M H, Park S U, Kang K H, Lee H S, Kim Y C; Hepatoprotective and free radical scavenging activities from Morus alba: Plant Med, 68, 932-940 (2002).
25. Andrabi S A, Spina M G, Lorenz P, Ebmeyer U, Wolf G, Horn T F; Oxyresveratrolm (trans-2,3′,4,5′-tetrahydroxystilbene) is neuroprotective and inhibits the apoptomic cell death in transient cerebral ischemia: Brain Res, 1017, 98-107 (2004).

These and other experimental studies have demonstrated the beneficial effects of resveratrol and oxyresveratrol in several health disorders. However, the mechanism of action that can explain their beneficial health benefits is not well understood. Furthermore, there is currently no available method of biochemically standardizing resveratrol or oxyresveratrol products currently sold as dietary supplements.

What is needed is a method of standardizing resveratrol products (including oxyresveratrol) based on its biochemical mechanism of action in order to produce uniform compositions and formulations for therapeutic uses.

### SUMMARY OF THE INVENTION

The present invention is directed to various methods for inhibiting the enzyme adenosine deaminase by the administration of standardized preparations of resveratrol related biochemicals. The standardized preparation will have a potency that is determined to be approximately equivalent to what is described in more detail herein as at least one enzyme inhibitory unit. Each enzyme inhibitory unit is equivalent to the concentration (μg/ml of the reaction mixture) of the preparation that inhibits adenosine deaminase activity by 50% under the enzyme assay conditions discussed under “Biochemical Mechanism of Action”. The standardized resveratrol or oxyresveratrol preparation, which inhibits adenosine deaminase activity can be used as described herein in therapeutic amounts to increase the tissue levels of adenosine. Adenosine lowers blood pressure and inhibits platelet aggregation (thrombosis).

The present invention also provides methods for preventing the degradation of adenosine into uric acid and oxidation products (superoxide radicals) by administering effective amounts of a standardized resveratrol preparation. By preventing the degradation of adenosine into oxidative by-products, standardized resveratrol or oxyresveratrol preparation can be used to prevent tissue damage to the heart or brain during the heart or stroke attacks caused by the depletion of cellular ATP and production of excessive amounts of superoxide radicals.

### Key Enzymatic Pathway Involved in Many Health Disorders

### E.g. Cell-Aging, Ischemic Heart Disease, Thrombosis, Hyperuricemia, Gout, Etc.

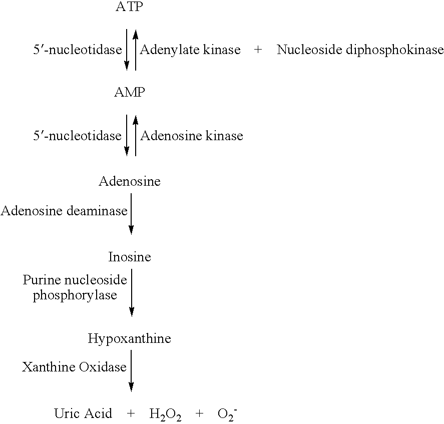

As illustrated above in the enzymatic pathway, the cellular ATP (adenosine 5′-triphosphate) metabolizes into various metabolites including adenosine, uric acid, and superoxide radicals. During a disease condition, the enzymatic pathway can be affected producing greater amounts of the metabolites, which are harmful to the body tissues. For example, during the process of heart attack or stroke, the cellular ATP is rapidly degraded into superoxide radicals that damage the heart or brain tissues. Also, in hyperuricemia or gout patients, there is an increase in blood levels of uric acid and superoxide radicals. Hyperuricemia patients suffer not only with gout but also from heart disease due to increased production of superoxide radicals.

The present invention further provides a method for biochemical standardization of resveratrol products. The amount of resveratrol or oxyresveratrol that inhibits adenosine deaminase activity by 50% (IC₅₀ value) is determined. This amount of the product is then set to be equivalent to one Enzyme Inhibitory Unit (EIU) of resveratrol.

### DETAILED DESCRIPTION OF THE INVENTION

### Labortory Research

The current invention pertains to methods for standardizing resveratrol products based upon their biochemical ability to inhibit adenosine deaminase enzyme activity. The invention also provides in various embodiments, methods of using the standardized preparations to treat diseases caused by an increase of blood uric acid levels (hyperuricemia) as seen in gout patients. Furthermore, the invention provides in various embodiments, methods of using the standardized resveratrol products to prevent tissue damage due to increased production of superoxide radicals as seen during the heart attack or stroke in ischemic artery diseases.

The following studies are undertaken to determine the mechanism of action that can support the therapeutic benefits of resveratrol products (including oxyresveratrol) and develop biochemically standardized preparations.
a. Biochemical mechanism of action responsible for its medical benefits to patients with heart disease, hyperuricemia and slow-downing of cell-aging, especially skin cells.
b. Biochemical standardization of products containing resveratrol and/or oxyresveratrol.

### Biochemical Mechanism of Action

Resveratrol (>99% pure) (3,4′,5-trihydroxytrans-stilbene) was purchased from Sigma-Aldrich Chemical Company, St. Louis, Mo. Solutions are made by dissolving 2 mg of resveratrol or the extract (Morus alba dried-form) per milliliter of ethanol (50%). Dose-response inhibitory effects of resveratrol on adenosine deaminase are carried out using the following spectrophotometric method described below.

### Adenosine Deaminase (ADA) Assay

The enzyme catalyzes the following reaction:

Adenosine+H₂O→Inosine+NH₃

The calf spleen adenosine deaminase (Specific Activity of 178 Units/mg protein) is purchased from Sigma Chemical Company, St Louis, Mo. The enzyme assay is carried out spectrophotometrically at 265 nm. The reaction mixture (in a 1-ml cuvette; diameter=1.0 cm) contains 0.9 ml of buffer (Tris-HCL buffer, 50 mM, pH 7.5), 25 μl of adenosine (2 mM), plus the enzyme, water and the extract to make 1 ml. The enzyme is added to start the reaction, and a decrease in absorbency at 265 nm is followed at 25° C. One unit of ADA activity is the amount of the enzyme that catalyzes 1 μmole of adenosine/min/ml of the reaction mixture.

### Inhibition of Adenosine Deaminase

The IC₅₀ values have been determined by examining the dose-response effects of both products. The IC₅₀ values represent the amount of the product per ml of the reaction mixture, producing the enzyme inhibition by 50%. In the enzyme assay, first a background reading is recorded on the chart for 1-2 min, followed by the addition of an appropriate amount of the enzyme to start the reaction, which is monitored spectrophotometrically. The amount of resveratrol or the mulberry extract added to the reaction mixture depends on the enzyme inhibition (10% to about 90%). FIGS. 1 and 2 show the IC₅₀ values determined from the above dose-response studies. These findings demonstrate that resveratrol and the extract containing oxyresveratrol inhibit adenosine deaminase with IC₅₀ values of 11.6 and 5.5 μg/ml respectively, suggesting that the extract, which contains oxyresveratrol demonstrates a much greater (2-fold) inhibition of the enzyme.

### Biochemical Standardization of Resveratrol Products Including Oxyresveratrol

The standardization of resveratrol products is typically carried out as the Enzyme Inhibitory Units (EIU) per mg of resveratrol or the mulberry extract containing resveratrol and oxyresveratrol. Typically, the IC₅₀ values are utilized for determination of Enzyme Inhibitory Units (EIU). The IC₅₀ value is equal to one EIU. The EIU value represents the potency of the products. For example, the IC₅₀ value (11.6 μg/ml) as determined for resveratrol (>99% pure, purchased from Sigma-Aldrich Chemical Company) above is equal to one EIU. Based on the IC₅₀ values, the EIU value of resveratrol is 86/mg, whereas, the EIU value for the mulberry extract, which contains oxyresveratrol is 182/mg. These findings demonstrate that the mulberry extract, which contains oxyresveratrol is 2-fold more biochemically effective than resveratrol.

### BRIEF DESCRIPTION OF THE FIGURES

FIG. 1 depicts the dose-response inhibition of adenosine deaminase by resveratrol. The half maximal inhibitory concentration of resveratrol (IC₅₀ value) is estimated. The IC₅₀ value represents the amount of the product per ml of the reaction mixture, producing the enzyme inhibition by 50%.

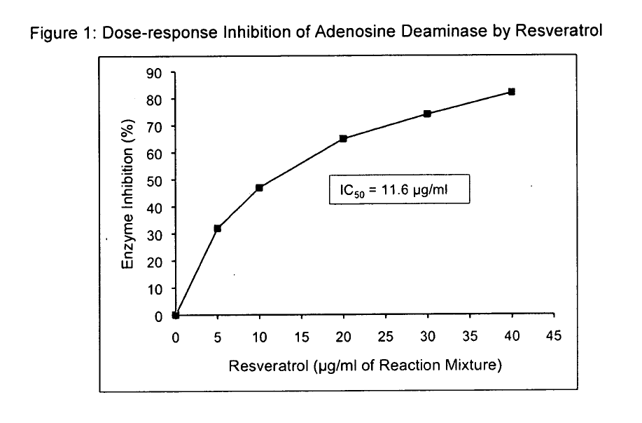

FIG. 2 depicts the dose-response inhibition of adenosine deaminase by the mulberry (Morus alba) wood extract containing oxyresveratrol. The half maximal inhibitory concentration of the extract (IC₅₀ value) is estimated. The IC₅₀ value represents the amount of the product per ml of the reaction mixture, producing the enzyme inhibition by 50%.

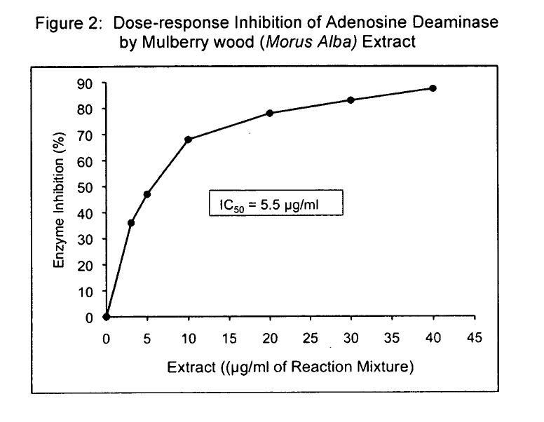

### THERAPEUTIC USES

### Heart Diseases, Hyperuricemia Etc.

The standardized resveratrol products can be utilized in a therapeutically effective amount in a host to treat, alleviate or prevent a disease condition. The term “therapeutically effective amount” refers to an amount of resveratrol product expressed as EIUs effective to “prevent,” “alleviate” or “treat” a disease or disorder in a subject. Generally, alleviation or treatment of a disease or disorder involves the lessening of one or more symptoms or medical problems associated with the disease or disorder. “Treating” or “treatment” or “alleviation” refers to both therapeutic treatment and prophylactic or preventative measures, wherein the object is to prevent or slow down (lessen) the targeted pathologic condition or disorder.

“Treating” or “treatment” or “alleviation” refers to both therapeutic treatment and prophylactic or preventive measures, wherein the object is to prevent or slow down (lesson) the targeted pathologic condition or disorder.

In addition, the standardized resveratrol or resveratrol products including mulberry extracts, by inhibiting adenosine deaminase, will prevent degradation of adenosine into harmful uric acid, superoxide radicals and hydrogen peroxide which can cause cell injury. Thus, preventing the production of these harmful degradation products will protect the host or subject from hyperuricemia, coronary artery disease including hypertension, and the cell-aging process of skin.

Furthermore, the standardized resveratrol or extracts containing structurally related resveratrol chemicals with high Enzyme Inhibitory Units of the current invention can be used to alleviate, treat or prevent conditions including but not limited to: hyperuricemia, gout, kidney stones, ischemic heart disease, or any other disease condition, where the cellular ATP degradation into superoxides radicals is increased.

1. The method for inhibiting the enzyme adenosine deaminase comprising administering a standardized resveratrol product or botanical extracts containing resveratrol and/or oxyresveratrol.

2. A method of claim 1, wherein the enzyme adenosine deaminase is inhibited by products containing resveratrol and/or oxyresveratrol.

3. A method for the biochemical standardization of resveratrol or oxyresveratrol products (a) determining the amount of the product that inhibits adenosine deaminase activity by 50%; and (b) setting the amount of the product determined in step (a) equivalent to one Enzyme Inhibitory Unit of the product sample.

4. The method of claim 3, wherein the standardized resveratrol or oxyresveratrol product has a potency equivalent to at least one Enzyme Inhibitory Unit (EIU), wherein each EIU is equivalent to the amount that inhibits adenosine deaminase by 50% (IC₅₀ value).

5. A method of claim 4 can be utilized to find out whether the enzyme-inhibitory activity of resveratrol or oxyresveratrol products is affected during the shelf-life storage.

6. The method of claim 4 to identify the most enzyme-inhibitory activity of the product with a highest EIU value and its uses thereof.

7. The method of claim 6, wherein the inhibition of adenosine deaminase activity results in a decrease in blood uric acid, superoxide radicals, and hydrogen peroxide in hyperuricemia or gout patients.

8. A method of claim 6, wherein the administration of an effective amount of a standardized resveratrol or oxyresveratrol preparation results in an increase in blood levels of adenosine in patients suffering with coronary artery disease (e.g. hypertension).

9. The method of claim 6, wherein the administration of an effective amount of a standardized resveratrol or oxyresveratrol preparation results in a decrease in superoxide radicals production in ischemic conditions of the body tissues (e.g. skin, heart and brain).

10. A pharmaceutical composition comprising a standardized resveratrol or oxyresveratrol preparation with one or more pharmaceutically acceptable diluents or excipients.
